(19) United States
(12) Patent Application Publication
(10) Pub. No.: US 2006/0025782 A1
(43) Pub. Date: Feb. 2, 2006

(54) Eliminator a personal appliance to assist in fecal evacuation for patients with rectal hernia (rectocele)

(76) Inventors: Steven Klein, Old Westbury, NY (US); Robert Couturier, Stamford, CT (US)

Correspondence Address: ROBERT COUTURIER, 1814 NEWFIELD AVENUE, STAMFORD, CT, 06903, US

(21) Appl. No.: 10/900,591

(22) Filed: Jul. 28, 2004

(51) Int. Cl. A61B 17/42

(52) U.S. Cl. 606/119

(57) ABSTRACT

The invention consists of a bent flat member, which, properly inserted in the vagina, may be used to help a patient affected with rectocele (hernia) eliminate fecal matter, which otherwise may remain in the pocket formed by the hernia, and cause medical problems. The instrument is washable and may be indefinitely re-used by the patient.

### FIELD OF THE INVENTION

In the medical field, the present invention concerns a personal appliance to assist in fecal evacuation for patients with rectal hernia (rectocele).

### BACKGROUND OF THE INVENTION

A person affected with rectocele accumulates fecal matter in the pocket formed by the hernia. Unless regularly removed, this matter becomes hardened, more difficult to eliminate, and a source of other medical problems. In female patients, it is possible to exert pressure on said matter through the walls of the vagina. An appliance, or “tool”, was conceived for that purpose.

### SUMMARY OF THE INVENTION

The “Eliminator” consists of a single rigid member, the approximate shape of a flat twelve inch ruler, but bent at some 45 degree angle some three inches from one end. It presents no sharp corners nor edges, and has a smooth non-absorbing surface. It may be washed with ordinary soap or detergent for multiple use.

### DESCRIPTION OF THE INVENTION

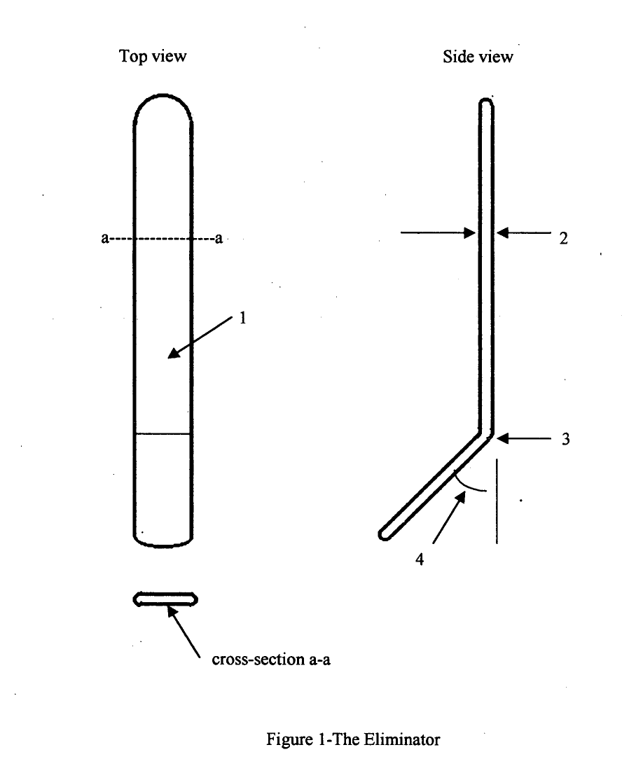

Reference is made to FIG. 1. The width of member (1) is approximately one inch. Its length is approximately twelve inches. Its thickness (2) shown in cross-section a-a, is selected depending on the chosen material, to be sufficient as to give a rigidity sufficient to sustain no bending at the corner (3) under a minimum torque of three pound-inches. All ends and edges are rounded off, for ease of insertion, and to prevent scarring of mucous membranes. All surfaces are smooth, non-porous, and washable. The instrument is indefinitely re-usable by the original owner.

1- A rigid member of, but not restricted to, one inch in width, and of, but not restricted to, twelve inches in length, and of a thickness selected according to the chosen material, in order to provide rigidity under expected flexing forces of no more than three pound-inches, said member (1) being bent near one-quarter of its length at an angle (4) of, but not restricted to, forty five degrees.

2- A rigid member as mentioned in claim 1 above, consisting of a) a uniform plastic material such as, but not restricted to, plexiglass, polycarbonates or other plastic materials, or b) a combination of metal plate suitably enrobed with aforementioned plastic materials in a). The relevance of the color of the material is solely related to pleasant appearance of the device, and hence may be any of the entire spectrum available.
